(19) United States
(12) Patent Application Publication
(10) Pub. No.: US 2026/0182897 A1
(43) Pub. Date: Jul. 2, 2026

(54) ELECTROCARDIOGRAPH SIGNAL MONITORING SYSTEM, ELECTROCARDIOGRAPH SIGNAL MONITORING METHOD AND ELECTROCARDIOGRAPH SIGNAL ANALYSIS SYSTEM

(71) Applicant: INDUSTRIAL TECHNOLOGY RESEARCH INSTITUTE, HSINCHU (TW)

(72) Inventor: Guo-Zua WU, Taichung City (TW)

(21) Appl. No.: 19/006,008

(22) Filed: Dec. 30, 2024

(30) Foreign Application Priority Data
Dec. 26, 2024 (TW) 113150808

(51) Int. Cl. A61B 5/349; A61B 5/00; A61B 5/308

(52) U.S. Cl. CPC A61B 5/349; A61B 5/308; A61B 5/746

(57) ABSTRACT

An electrocardiograph signal monitoring system, comprising at least three electrodes, a lead analysis circuit and a lead generation circuit. The lead analysis circuit is coupled to the electrodes, and is configured to calculate multiple first electrocardiograph signals according to multiple electrode signals. The lead generation circuit is configured to generate multiple second electrocardiograph signals according to the first electrocardiograph signals, and integrate the first electrocardiograph signals and the second electrocardiograph signals into a twelve-lead electrocardiograph data. The lead generation circuit comprises at least one signal generator comprising multiple data filters, the data filters are configured to capture multiple signal characteristics corresponding to different sampling frequencies in the first electrocardiograph signals, so that the signal generator generates the second electrocardiograph signals.

### BACKGROUND

### Technical Field

The technical field relates to an electrocardiograph signal monitoring system, an electrocardiograph signal monitoring method and an electrocardiograph signal analysis system.

### Description of Related Art

“Electrocardiograph” (ECG or EKG) is a type of test data used to determine the state of heart rhythm. During an ECG detection, multiple electrodes are placed at various locations on the subject's body to capture tiny current signals on the body's surface caused by the heartbeat. The detection device can read these current signals, organize and record the signals as signal waveforms, which constitute the electrocardiograph.

### SUMMARY

One embodiment of the present disclosure is an electrocardiograph signal monitoring system, comprising at least three electrodes, a lead analysis circuit and a lead generation circuit. The at least three electrodes are configured to obtain a plurality of electrode signals. The lead analysis circuit is coupled to the at least three electrodes, and is configured to calculate a plurality of first electrocardiograph signals according to the plurality of electrode signals. The lead generation circuit is coupled to the lead analysis circuit, and is configured to generate a plurality of second electrocardiograph signals according to the first electrocardiograph signals. The lead generation circuit is further configured to integrate the plurality of first electrocardiograph signals and the plurality of second electrocardiograph signals into a twelve-lead electrocardiograph data. The lead generation circuit comprises at least one signal generator. The at least one signal generator comprises a plurality of data filters, the plurality of data filters are configured to capture a plurality of signal characteristics corresponding to different sampling frequencies in the plurality of first electrocardiograph signals, so that the at least one signal generator generates the plurality of second electrocardiograph signals.

Another embodiment of the present disclosure is an electrocardiograph signal monitoring method, comprising: receiving, by at least three electrodes, a plurality of electrode signals; calculating, by a lead analysis circuit, a plurality of first electrocardiograph signals according to the plurality of electrode signals; capturing, by a plurality of data filters of at least one signal generator, a plurality of signal characteristics corresponding to different sampling frequencies in the plurality of first electrocardiograph signals, so that the at least one signal generator generates a plurality of second electrocardiograph signals; and integrating the plurality of first electrocardiograph signals and the plurality of second electrocardiograph signals into a twelve-lead electrocardiograph data.

Another embodiment of the present disclosure is an electrocardiograph signal analysis system, comprising a first electrocardiograph analysis circuit and a second electrocardiograph analysis circuit. The first electrocardiograph analysis circuit is configured to receive a plurality of electrode signals from at least three electrodes, and calculate a plurality of first electrocardiograph signals according to the plurality of electrode signal. When the first electrocardiograph analysis circuit determine the plurality of first electrocardiograph signals matches a first waveform characteristic, the first electrocardiograph analysis circuit is configured to generate a first warning signal. The second electrocardiograph analysis circuit is coupled to the first electrocardiograph analysis circuit, and is configured to generate a plurality of second electrocardiograph signals according to the first electrocardiograph signals when the plurality of first electrocardiograph signals does not match the first waveform characteristic. The second electrocardiograph analysis circuit is further configured to integrate the plurality of first electrocardiograph signals and the plurality of second electrocardiograph signals into a twelve-lead electrocardiograph data. The second electrocardiograph analysis circuit is further configured to compare the twelve-lead electrocardiograph data with a second waveform characteristic. The second electrocardiograph analysis circuit generates a second warning signal when the plurality of first electrocardiograph signals matches the second waveform characteristic. The first waveform characteristic and the second waveform characteristic correspond to different waveform segments respectively.

### BRIEF DESCRIPTION OF THE DRAWINGS

The present disclosure can be more fully understood by reading the following detailed description of the embodiment, with reference made to the accompanying drawings as follows:

FIG. 1 is a schematic diagram of the electrocardiograph detection.

FIG. 2 is a schematic diagram of an electrocardiograph signal monitoring system in some embodiments of the present disclosure.

FIG. 3 is a schematic diagram of a waveform of a lead signal in electrocardiograph detection.

FIG. 4 is a flowchart illustrating an electrocardiograph signal monitoring method in some embodiments of the present disclosure.

FIG. 5A is a schematic diagram of the structure of a signal generator in some embodiments of the present disclosure.

FIG. 5B is a schematic diagram of the computational block in some embodiments of the present disclosure.

FIG. 5C is a schematic diagram of the data construction unit in some embodiments of the present disclosure.

### DETAILED DESCRIPTION

For the embodiment below is described in detail with the accompanying drawings, embodiments are not provided to limit the scope of the present disclosure. Moreover, the operation of the described structure is not for limiting the order of implementation. Any device with equivalent functions that is produced from a structure formed by a recombination of elements is all covered by the scope of the present disclosure. Drawings are for the purpose of illustration only, and not plotted in accordance with the original size.

It will be understood that when an element is referred to as being “connected to” or “coupled to”, it can be directly connected or coupled to the other element or intervening elements may be present. In contrast, when an element to another element is referred to as being “directly connected” or “directly coupled,” there are no intervening elements present. As used herein, the term “and/or” includes an associated listed items or any and all combinations of more.

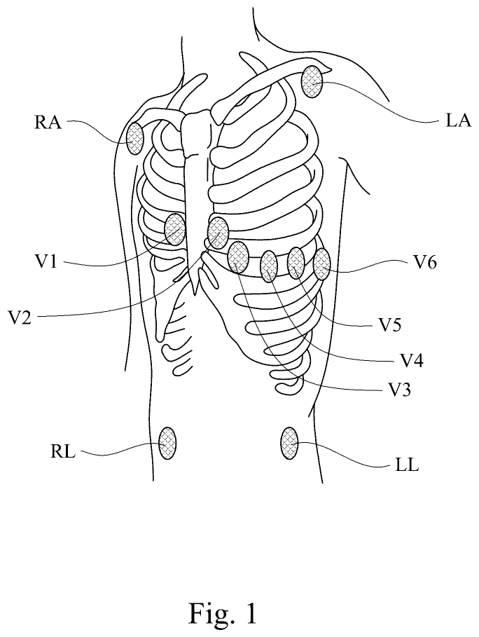

FIG. 1 is a schematic diagram of an electrocardiograph detection (ECG detection). During the ECG detection, multiple electrodes are arranged on the subject's body, as the electrode LA, the electrode RA, the electrodes V1-V6, the electrode RL and the electrode LL shown in FIG. 1. The signals recorded by each pair of two electrodes are called a “lead”. In a complete ECG detection, ten electrodes are used to record twelve lead signals (referred to as “twelve-lead”).

However, the twelve-lead detection requires specialized equipment, and the configuration of ten electrodes is not suitable for long-term (e.g., 24 hours) monitoring. Therefore, the present disclosure uses a small number of electrodes (e.g., three electrodes LL, LA, RA) to capture signals and uses a neural network for analysis to generate a twelve-lead signal waveform. In addition, the present disclosure further improves the signal generation method and circuit architecture to enhance the computational accuracy.

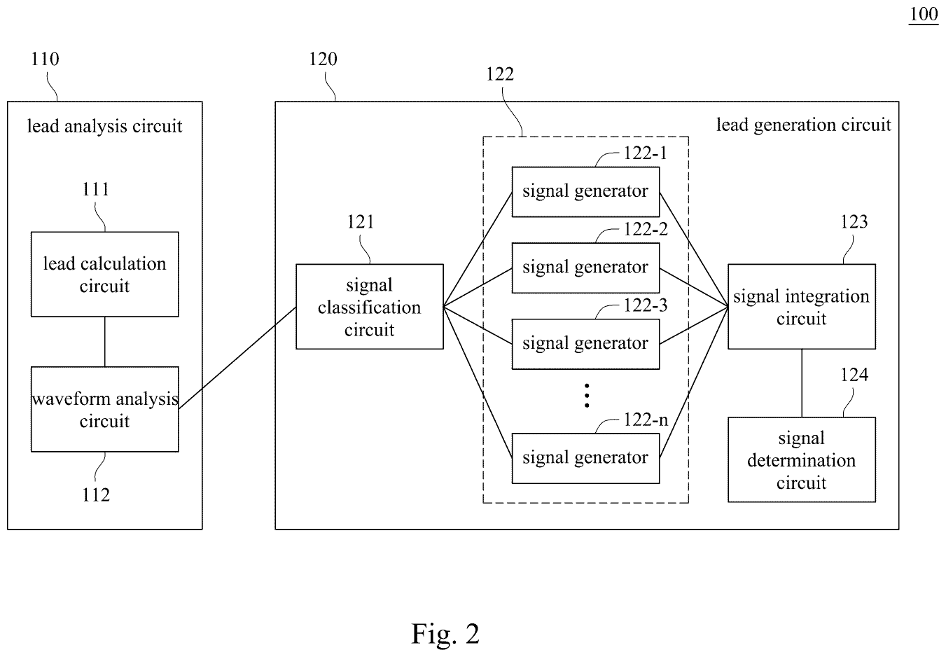

FIG. 2 is a schematic diagram of an electrocardiograph signal monitoring system 100 in some embodiments of the present disclosure. The electrocardiograph signal monitoring system 100 includes at least three electrodes (e.g., as electrodes LL, LA, RA shown in FIG. 1), a lead analysis circuit 110 and a lead generation circuit 120. The electrocardiograph signal monitoring system 100 is configured to monitor the electrocardiograph data of the subject, and compute/estimate more electrocardiograph characteristic according to the monitored data to generate an electrocardiograph data for review. In some other embodiments, the electrocardiograph signal monitoring system 100 may also be implemented to an electrocardiograph signal analysis system, which is configured to analyze the monitored data to obtain signal characteristics and perform multiple phases of comparison in sequence to generate corresponding notifications or warning signals.

In one embodiment, three electrodes LL, LA, RA are configured to obtain multiple electrode signals, such as voltage or current. The lead analysis circuit 110 is coupled to the electrodes LL, LA, RA, and is configured to calculate/estimate multiple lead signals according to the electrode signals. As mentioned above, the change of the electrode signal recorded by the circuit composed of two electrodes is called a “lead signal”. Therefore, by capturing electrode signals through multiple electrodes LL, LA, and RA, a portion of the twelve-lead electrocardiograph data can be directly obtained. These multiple lead signals that can be directly obtained are referred to as “first electrocardiograph signals”.

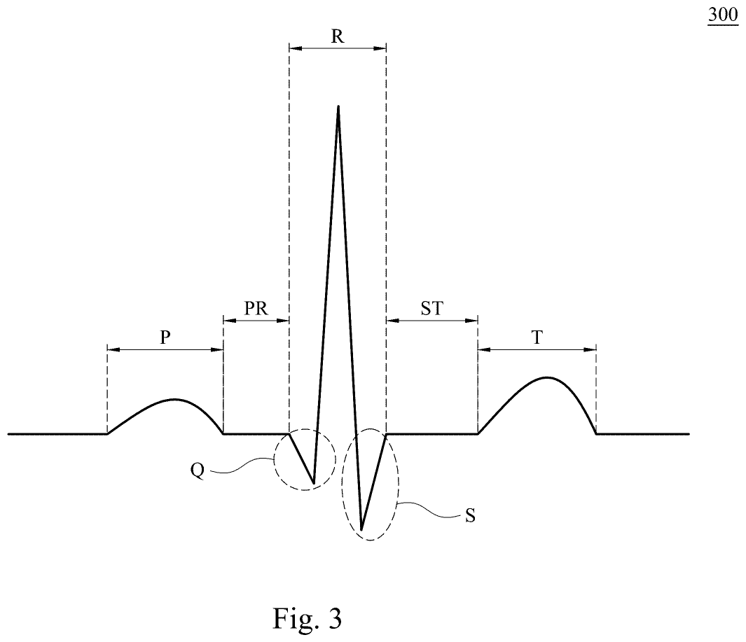

Specifically, the lead analysis circuit 110 includes a lead calculation circuit 111. The lead calculation circuit 111 calculates six lead signals (i.e., the first electrocardiograph signals) according to at least three received electrode signals and the lead definition of the electrocardiograph. The six lead signals include a first limb lead I, a second limb lead II, a third limb lead III, a lead aVR, a lead aVL and a lead aVF. The definitions of the above six lead signals are as follows:
Lead I: LA-RA
Lead II: LL-RA
Lead III: LL-LA
Lead aVR: −(I+II)/2
Lead aVL: I−(II/2)
Lead aVF: II−(I/2)
In one embodiment, the lead analysis circuit 110 further includes a waveform analysis circuit 112. The waveform analysis circuit 112 is coupled to the lead calculation circuit 111, and is configured to analyze whether the first electrocardiograph signal(s) have specific waveform characteristics. Referring to FIG. 3, FIG. 3 shows a waveform of a lead signal 300 in the ECG detection. According to the changing trend of the signal, the lead signal 300 can be divided into multiple waveform segments P, Q, R, S, T, PR, ST. The definition and characteristics of the waveform segments P, Q, R, S, T, PR, ST can be defined in the lead analysis circuit 110 in advance. For example, the definition of “waveform segment Q” is the segment of “the first downward transition in the waveform”. In one embodiment, the lead analysis circuit 110 determines whether any one of the first electrocardiograph signals matches to a “large Q wave” (i.e., the downward turning length of the waveform segment Q is greater than a predetermined value). If one of the first electrocardiograph signals matches to the “large Q wave”, a first warning signal (e.g., warning of myocardial infarction) will be generated.

The lead generation circuit 120 is coupled to the lead analysis circuit 110 to receive the first electrocardiograph signals. The lead generation circuit 120 includes at least one signal generator 122, so as to generate multiple lead signals (the other six lead signals V1-V6 in the twelve leads) according to the first electrocardiograph signals. The lead generation circuit 120 may be a neural network, such as a generative neural network, and its model architecture will be described in detail in subsequent paragraphs. The other six lead signals (e.g., leads V1-V6) calculated by the lead generation circuit 120 are referred to as “second electrocardiograph signals”. The lead generation circuit 120 can integrate the first electrocardiograph signals and the second electrocardiograph signals into a “twelve-lead electrocardiograph data”.

In one embodiment, the lead generation circuit 120 includes a signal classification circuit 121, at least one signal generator 122, a signal integration circuit 123 and a signal determination circuit 124, and the number of the signal generator 122 can be plural, such as the signal generators 122-1˜122-n shown in FIG. 2, which have different computational parameter(s). The signal classification circuit 121 is coupled to the waveform analysis circuit 112 and the signal generators 122-1˜122-n, and is configured to analyze the waveform of the first electrocardiograph signals, and classify the first electrocardiograph signals. According to the classification result, the signal classification circuit 121 will select one of the signal generators 122-1˜122-n to generate the second electrocardiograph signals.

The signal integration circuit 123 is coupled to the signal generators 122-1˜122-n, and is configured to receive the second electrocardiograph signals generated by the signal generators 122-1˜122-n, and integrate the first electrocardiograph signals and the second electrocardiograph signals into a twelve-lead electrocardiograph data. The signal determination circuit 124 is configured to analyze the twelve-lead electrocardiograph data, such as compare the twelve-lead electrocardiograph data with a second waveform characteristic (e.g., special waveform characteristics of the waveform segments R, Q, ST). When the twelve-lead electrocardiograph data matches the second waveform characteristic, the signal determination circuit 124 is configured to generate a second warning signal.

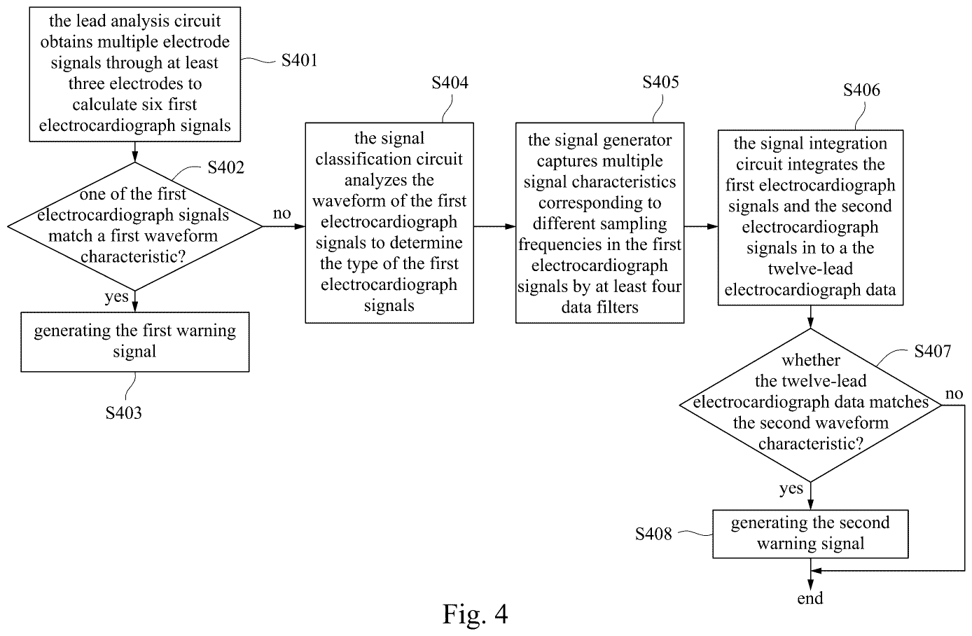

The operation of the electrocardiograph signal monitoring system 100 is described by taking the flow chart shown in FIG. 4 as an example. In step S401, the lead analysis circuit 110 obtains multiple electrode signals through at least three electrodes (e.g., electrodes LL, LA, RA), and calculates six lead signals, such as lead signal I, II, III, aVR, aVL, aVF (i.e., the first electrocardiograph signals), according to the definition of lead signal.

In step S402, the waveform analysis circuit 112 of the lead analysis circuit 110 determine whether one of the first electrocardiograph signals match a first waveform characteristic. In one embodiment, “first waveform characteristic” includes a Q wave (the waveform segment Q) waveform characteristic or a downward waveform characteristic (e.g., the downward turning length of Q wave is greater than a predetermined value). The lead analysis circuit 110 compares a corresponding segment (e.g., as the waveform segment Q shown in FIG. 3) of the first electrocardiograph signals with the first waveform characteristic to determine whether they match.

In step S403, if one of the first electrocardiograph signals matches the first waveform characteristic, the lead analysis circuit 110 generates the first warning signal, and transmits the first warning signal to a terminal device (e.g., a display panel of detection equipment, or a mobile phone of the subject).

If all of the first electrocardiograph signals do not match the first waveform characteristic, at this time, the lead analysis circuit 110 transmits the first electrocardiograph signals to the lead generation circuit 120, so as to use the first electrocardiograph signals to generate the other six lead signals (i.e., the second electrocardiograph signals).

Specifically, in step S404, the signal classification circuit 121 analyzes the waveform of the first electrocardiograph signals to determine the type of the first electrocardiograph signals. According to the analysis results, the signal classification circuit 121 selects one of the signal generators 122-1˜122-n to generate the other six lead signals (i.e., the second electrocardiograph signals). In other words, the signal classification circuit 121 defines the waveform characteristics of the first electrocardiograph signals in advance to classify the first electrocardiograph signals, and different types of the first electrocardiograph signals correspond to different signal generators 122-1˜122-n.

In some embodiments, the signal classification circuit 121 analyzes the waveform of “lead signal I (the first limb lead signal)” and the waveform of “lead signal II (the second limb lead signal)” (i.e., analyzes two of the six lead signals), so as to determine the type of the first electrocardiograph signals.

In one embodiment, the signal classification circuit 121 analyzes the waveform of the first limb lead signal and the waveform of the second limb lead signal to determine the heartbeat cycle in the first electrocardiograph signals. Then, it selects the corresponding signal generator according to the heartbeat cycle. In other words, the computational parameter(s) in the selected signal generator corresponds to the duration of the heartbeat cycle. The correspondence between the computational parameter(s) and the heartbeat cycle can be set in advance in the signal classification circuit 121. For example, when the heartbeat cycle is “60 times per minute”, the signal classification circuit 121 selects the signal generator 122-1. When the heartbeat cycle is “61 times per minute”, the signal classification circuit 121 selects another signal generator 122-2, and so on.

The analysis target of the signal classification circuit 121 is not restricted to the heartbeat cycle. In some other embodiments, the signal classification circuit 121 can also analyze the frequency, amplitude or a specific waveform segment to select one of the corresponding signal generators 122-1˜122-n. In addition, in some embodiments, the lead generation circuit 120 may be configured with only one single signal generator. That is, the lead generation circuit 120 may not analyze the first electrocardiograph signals, but may use a fixed signal generator to generate the second electrocardiograph signals.

In step S405, after selecting one of the signal generators 122-1˜122-n, the selecting one of the signal generators 122-1˜122-n captures multiple signal characteristics corresponding to different sampling frequencies in the first electrocardiograph signals by multiple internal data filters. For example, capturing the signal characteristics corresponding to different time scales or corresponding to different frequency ranges. Next, the selecting one of the signal generators 122-1˜122-n also generates the second electrocardiograph signals according to the captured signal characteristics. In one embodiment, the number of the data filters is at least four to cover a wider range of characteristics, but the number of the data filters can be adjusted according to requirements.

In step S406, the signal integration circuit 123 integrates the first electrocardiograph signals and the second electrocardiograph signals in to the twelve-lead electrocardiograph data.

In step S407, the signal determination circuit 124 compares the twelve-lead electrocardiograph data with the second waveform characteristic to determine whether the twelve-lead electrocardiograph data matches the second waveform characteristic. The second waveform characteristic and the first waveform characteristic correspond to different waveform segments. For example, the first waveform characteristic includes a Q wave waveform characteristic, and the second waveform characteristic includes a ST waveform characteristic or a R wave waveform characteristic.

In step S408, if the twelve-lead electrocardiograph data matches the second waveform characteristic, the signal determination circuit 124 generates the second warning signal to the terminal device (e.g., a display panel of detection equipment, or a mobile phone of the subject). In other words, the electrocardiograph signal monitoring system 100 analyzes the electrocardiograph signal in multiple stages, and each analysis targets a different waveform segment. Accordingly, possible abnormalities in the electrocardiograph signal will be more efficiently identified for confirmation by the subject.

In the aforementioned embodiment, the electrocardiograph signal monitoring system 100 calculates the first electrocardiograph signals by the lead analysis circuit 110, and estimates the second electrocardiograph signals by the lead generation circuit 120. Since the lead analysis circuit 110 is further configured to compare the first electrocardiograph signals with the first waveform characteristic, the lead generation circuit 120 is configured to compare the twelve-lead electrocardiograph data with the second waveform characteristic, in some embodiments, the lead analysis circuit 110 may also be referred to as a first electrocardiograph analysis circuit, and the lead generation circuit 120 may be referred to as a second electrocardiograph analysis circuit.

In addition, after the determination in the aforementioned steps S402 and S407, the electrocardiograph signal monitoring system 100 can also organize the determination results (e.g., the first electrocardiograph signals, the second electrocardiograph signals, the first warning signal or the second warning signal) into a detection data and provide the detection data to an external device (e.g., databases, servers, etc.). In some other embodiments, the electrocardiograph signal monitoring system 100 can also compare the calculated twelve-lead electrocardiograph data with another detection lead data (e.g., a twelve-lead electrocardiograph actually detected by twelve electrodes). According to the comparison results, the twelve-lead electrocardiograph data is input into the signal generator 122 as training data.

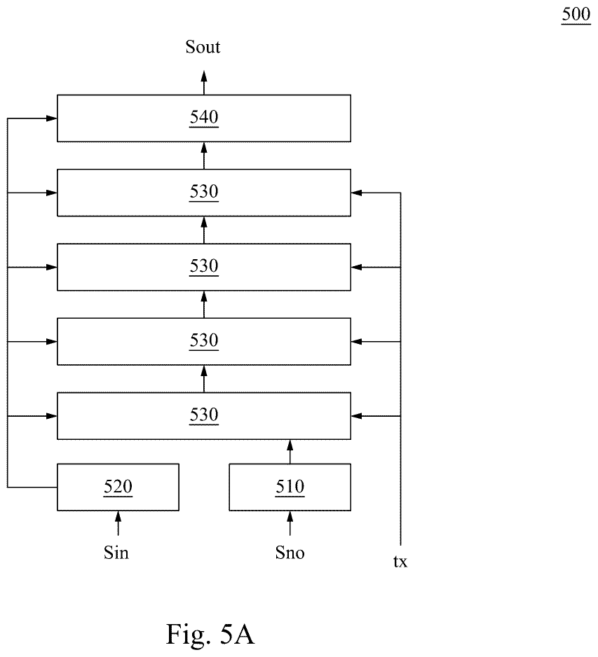

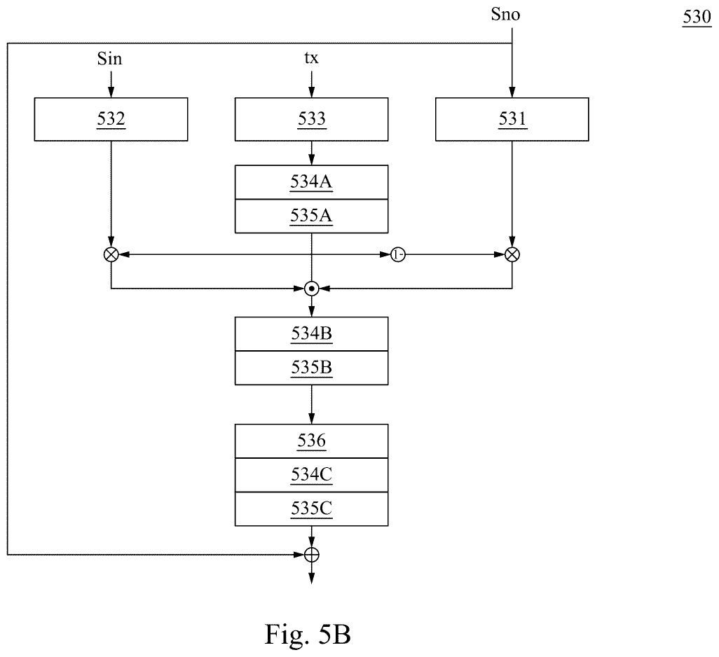

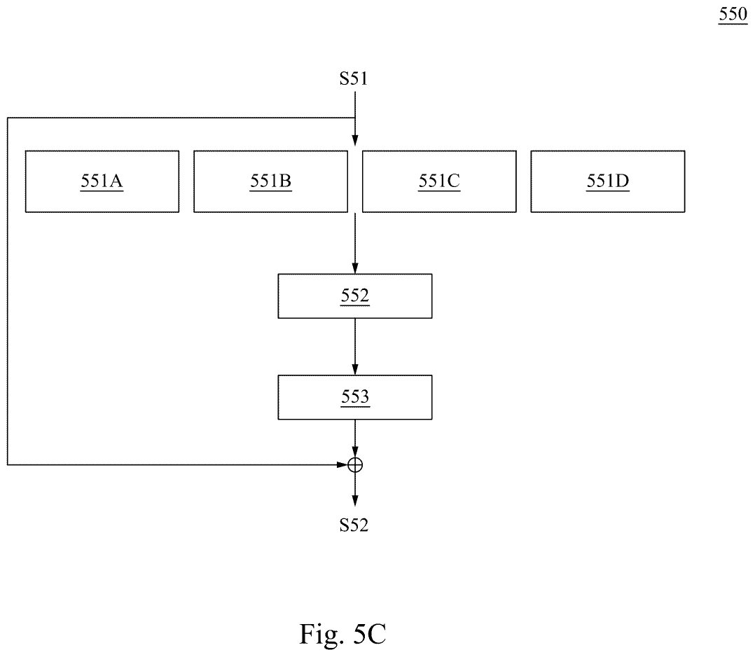

The following describes the architecture features of the neural network of the signal generator 122. Referring to FIG. 5A, it shows a schematic diagram of the architecture of a signal generator 500 according to some embodiments of the present disclosure, which can be used to implement any one of the signal generators 122, 122-1 to 122-n in FIG. 2.

The signal generator 500 is a type of a Generative Adversarial Network (GAN), such as an Augmented Generative Neural Network Models, or a Denoising Diffusion Probabilistic Model Generative Neural Network. The signal generator 500 includes a convolutional layer 510, a convolutional layer 520, multiple computational blocks 530 and a convolutional layer 540. In one embodiment, the signal generator 500 can use multiple known first limb lead signals I and second limb lead signals II as training data to establish computational parameter(s) of each convolutional layer or computational block in the signal generator 500.

The convolutional layer 510 is configured to receive an input data Sin (e.g., the above first electrocardiograph signals), and the convolutional layer 520 is configured to generate a noise Sno (e.g., random vector) required for the computation. Data generated by the convolutional layers 510, 520 computation is output to the computational block 530 and the convolutional layer 540. Each of the computational blocks further includes computation structures such as a convolutional layer, an activation layer and a normalization layer, and computes the input data Sin and/or the noise Sno according to the time parameter tx. The output data Sout(e.g., the second electrocardiograph signals) final generated by the convolutional layer 540. Since one of the ordinary skills in the art can understand the operation of the convolutional layer and blocks in the neural network, so it will not be described here in detail.

FIG. 5B is a schematic diagram of the computational block 530 in some embodiments of the present disclosure, which can be used to implement anyone of the computational blocks 530 as shown in FIG. 5A. The computational block 530 includes a data construction unit 531, a data construction unit 532, a positional encoding unit 533, multiple convolutional layers 534A-534C, multiple activation units 535A-535C (e.g., Rectified Linear Function, ReLU) and a deconvolutional layer 536.

The data construction units 531, 532 may be HNF modules (Hierarchical Normalizing Flow). The HNF module can be implemented by algorithms or hardware circuits to model and analyze the features structure of signals. The HNF module can process complex data distributions, learn key features in the signal layer by layer, and can also generate simulated data. The data construction unit 531, 532 is configured to respectively receive the noise Sno and the input data Sin to compute, so as to obtain the key features or simulate data.

The positional encoding unit 533 is configured to receive the time parameter tx to generate a time vector representing the location feature. Since one of ordinary skill in the art can understand the data output by the data construction unit and the operation of the convolutional layer, the activation function, and the deconvolutional layer, so it will not be described here in detail.

FIG. 5C is an internal schematic diagram of the data construction unit 550 in some embodiments of the present disclosure, which can be used to implement anyone of the data construction units 531, 532 as shown in FIG. 5B. The data construction unit 550 includes at least four data filters 551A-551D, a depth filter 552 and a convolutional layer 553. The data filters 551A-551D respectively correspond to different sampling frequencies (i.e., time scales, and/or feature ranges), and thus can capture multiple signal characteristics corresponding to different sampling frequencies in the input signal S51 (e.g., the input data Sin or the noise Sno), so that the signal generator 500 generates the second electrocardiograph signals accordingly.

Specifically, in one embodiment, the data filters 551A-551D are different convolutional layers, and have different sizes of the convolution kernels, but have the same number of convolution kernels. For example, the convolution kernel size of the data filter 551A is 3×1, the convolution kernel size of the data filter 551B is 15×1, the convolution kernel size of the data filter 551C is 9×1, the convolution kernel size of the data filter 551D is 5×1, and the number of convolution kernels of all of the data filters 551A-551D is 20. Through different convolutional layers, the data filters 551A-551D can capture the characteristics of the signal (e.g., the input data Sin or the noise Sno) with different “time scales” or “feature ranges”.

The depth filter 552 is configured to receive multiple filter signals (i.e., the captured signal characteristics) output by the data filters 551A-551D, and is configured to integrate (e.g., concatenate) these filter signals into the characteristic signal. Next, the depth filter 552 further captures the characteristic(s) in the characteristic signal to generate a depth filter signal. In one embodiment, the depth filter 552 can be a convolutional layer. The convolution kernel size of the depth filter 552 is the same as that of one of the data filters 551A-551D (e.g., 9×1). The number of the convolution kernels of the depth filter 552 may be the sum of the number of the convolution kernels of the data filters 551A-551D, but the present disclosure is not limited thereto.

In one embodiment, the depth filter signal output by the depth filter 552 will first undergo a channel split. One part will be processed with instance normalization, while the other part will not undergo normalization. The signals from both parts will then be processed with Leaky ReLU and then input to the convolutional layer 553 to generate the output signal S52.

Accordingly, since the data construction unit 550 of the signal generator 500 uses multiple different data filters to capture the characteristics of the input signal (e.g., the first electrocardiograph signals, the input data Sin or the noise Sno), the characteristics of the signal can be analyzed more completely and accurately. In addition, the data construction unit 550 can further integrate the signal and capture characteristics again by the depth filter 552. Therefore, the integrity of signal analysis can be further enhanced to ensure that the generated second electrocardiograph signals reflect the actual conditions.

The electrocardiograph signal monitoring/analysis system provided by the present disclosure can analyze the electrocardiograph signal in multiple stages to find/identify possible abnormalities in the electrocardiograph signal for different waveform segments. In addition, by using multiple different data filters in the generative neural network to capture/extract characteristic(s) at different frequencies from the input signal, the electrocardiograph signal monitoring system can analyze and generate complete twelve-lead electrocardiograph data according to a portion of the detection signal (i.e., the electrocardiograph signals actually detected). Overall, the electrocardiograph signal monitoring/analysis system and the electrocardiograph signal monitoring/analysis method provided by the present disclosure are not only easy to implement, but also can improve the accuracy of signal analysis, monitoring and prediction without the need for complicated professional instruments.

The elements, method steps, or technical features in the foregoing embodiments may be combined with each other, and are not limited to the order of the specification description or the order of the drawings in the present disclosure.

It will be apparent to those skilled in the art that various modifications and variations can be made to the structure of the present disclosure without departing from the scope or spirit of the present disclosure. In view of the foregoing, it is intended that the present disclosure cover modifications and variations of this present disclosure provided they fall within the scope of the following claims.

What is claimed is:

1. An electrocardiograph signal monitoring system, comprising:
at least three electrodes configured to obtain a plurality of electrode signals;
a lead analysis circuit coupled to the at least three electrodes, and configured to calculate a plurality of first electrocardiograph signals according to the plurality of electrode signals; and
a lead generation circuit coupled to the lead analysis circuit, configured to generate a plurality of second electrocardiograph signals according to the first electrocardiograph signals, and configured to integrate the plurality of first electrocardiograph signals and the plurality of second electrocardiograph signals into a twelve-lead electrocardiograph data;
wherein the lead generation circuit comprises at least one signal generator, the at least one signal generator comprises a plurality of data filters, the plurality of data filters are configured to capture a plurality of signal characteristics corresponding to different sampling frequencies in the plurality of first electrocardiograph signals, so that the at least one signal generator generates the plurality of second electrocardiograph signals.

2. The electrocardiograph signal monitoring system of claim 1, wherein the at least one signal generator comprises a plurality of signal generators, and the lead generation circuit further comprises a signal classification circuit, which is configured to analyze a plurality of waveforms of the plurality of first electrocardiograph signals to select one of the plurality of signal generators to generate the plurality of second electrocardiograph signals.

3. The electrocardiograph signal monitoring system of claim 2, wherein the signal classification circuit analyzes a waveform of a first limb lead signal and a waveform of a second limb lead signal in the plurality of first electrocardiograph signals to determine a heartbeat cycle in the plurality of first electrocardiograph signals.

4. The electrocardiograph signal monitoring system of claim 3, wherein the plurality of signal generators are a type of generative neural network.

5. The electrocardiograph signal monitoring system of claim 1, wherein a number of the plurality of data filters is at least four.

6. The electrocardiograph signal monitoring system of claim 1, wherein the at least one signal generator comprises a depth filter, the depth filter is configured to receive a plurality of filter signals output by the plurality of data filters to integrate the plurality of filter signals into a characteristic signal; and
wherein the depth filter is further configured to capture a characteristic in the characteristic signal so that the at least one signal generator generates the plurality of second electrocardiograph signals.

7. The electrocardiograph signal monitoring system of claim 1, wherein the lead generation circuit is configured to compare the plurality of first electrocardiograph signals with a downward waveform characteristic, and generate a first warning signal when one of the plurality of first electrocardiograph signals matches the downward waveform characteristic.

8. An electrocardiograph signal monitoring method, comprising:
receiving, by at least three electrodes, a plurality of electrode signals;
calculating, by a lead analysis circuit, a plurality of first electrocardiograph signals according to the plurality of electrode signals;
capturing, by a plurality of data filters of at least one signal generator, a plurality of signal characteristics corresponding to different sampling frequencies in the plurality of first electrocardiograph signals, so that the at least one signal generator generates a plurality of second electrocardiograph signals; and
integrating the plurality of first electrocardiograph signals and the plurality of second electrocardiograph signals into a twelve-lead electrocardiograph data.

9. The electrocardiograph signal monitoring method of claim 8, wherein the at least one signal generator comprises a plurality of signal generators, and the electrocardiograph signal monitoring method further comprises:
analyzing, by a signal classification circuit, a plurality of waveforms of the plurality of first electrocardiograph signals to select one of the plurality of signal generators to generate the plurality of second electrocardiograph signals.

10. The electrocardiograph signal monitoring method of claim 9, wherein analyzing the plurality of waveforms of the plurality of first electrocardiograph signals comprises:
analyzing a waveform of a first limb lead signal and a waveform of a second limb lead signal in the plurality of first electrocardiograph signals to determine a heartbeat cycle in the plurality of first electrocardiograph signals.

11. The electrocardiograph signal monitoring method of claim 10, wherein the plurality of signal generators are a type of generative neural network.

12. The electrocardiograph signal monitoring method of claim 8, wherein a number of the plurality of data filters is at least four, and the electrocardiograph signal monitoring method further comprises:
receiving, by a depth filter of the at least one signal generator, a plurality of filter signals output by the plurality of data filters to integrate the plurality of filter signals into a characteristic signal; and
capturing, by the depth filter, a characteristic in the characteristic signal so that the at least one signal generator generates the plurality of second electrocardiograph signals.

13. The electrocardiograph signal monitoring method of claim 8, further comprising:
comparing the plurality of first electrocardiograph signals with a downward waveform characteristic; and
generating a first warning signal when one of the plurality of first electrocardiograph signals matches the downward waveform characteristic.

14. An electrocardiograph signal analysis system, comprising:
a first electrocardiograph analysis circuit configured to receive a plurality of electrode signals from at least three electrodes, and calculate a plurality of first electrocardiograph signals according to the plurality of electrode signals, wherein when the first electrocardiograph analysis circuit determine the plurality of first electrocardiograph signals matches a first waveform characteristic, the first electrocardiograph analysis circuit is configured to generate a first warning signal; and
a second electrocardiograph analysis circuit coupled to the first electrocardiograph analysis circuit, configured to generate a plurality of second electrocardiograph signals according to the first electrocardiograph signals when the plurality of first electrocardiograph signals does not match the first waveform characteristic, and integrate the plurality of first electrocardiograph signals and the plurality of second electrocardiograph signals into a twelve-lead electrocardiograph data;
wherein the second electrocardiograph analysis circuit is further configured to compare the twelve-lead electrocardiograph data with a second waveform characteristic, the second electrocardiograph analysis circuit generates a second warning signal when the plurality of first electrocardiograph signals matches the second waveform characteristic;
wherein the first waveform characteristic and the second waveform characteristic correspond to different waveform segments respectively.

15. The electrocardiograph signal analysis system of claim 14, wherein the first waveform characteristic comprises a Q wave characteristic, and the second waveform characteristic comprises an ST wave characteristic or an R wave characteristic.

16. The electrocardiograph signal analysis system of claim 14, wherein the second electrocardiograph analysis circuit comprises at least one signal generator, the at least one signal generator comprises at least four data filters, the at least four data filters are configured to capture a plurality of signal characteristics corresponding to different sampling frequencies in the plurality of first electrocardiograph signals, so that the at least one signal generator generates the plurality of second electrocardiograph signals.

17. The electrocardiograph signal analysis system of claim 16, wherein the at least one signal generator comprises a plurality of signal generators, and the second electrocardiograph analysis circuit further comprises a signal classification circuit, which is configured to analyze a plurality of waveforms of the plurality of first electrocardiograph signals to select one of the plurality of signal generators to generate the plurality of second electrocardiograph signals.

18. The electrocardiograph signal analysis system of claim 17, wherein the signal classification circuit analyzes a waveform of a first limb lead signal and a waveform of a second limb lead signal in the plurality of first electrocardiograph signals to determine a heartbeat cycle in the plurality of first electrocardiograph signals.

19. The electrocardiograph signal analysis system of claim 16, wherein the at least one signal generator comprises a depth filter, the depth filter is configured to receive a plurality of filter signals output by the at least four data filters to integrate the plurality of filter signals into a characteristic signal; and
wherein the depth filter is further configured to capture a characteristic in the characteristic signal so that the at least one signal generator generates the plurality of second electrocardiograph signals.
